(19) United States
(12) Patent Application Publication
(10) Pub. No.: US 2010/0312010 A1
(43) Pub. Date: Dec. 9, 2010

(54) Process for the Preparation of (S)-Pregabalin

(75) Inventors: Amit Anant Chavan, Kalwa (IN); Shashikant Balasaheb Padwal, Pune (IN); Ashutosh Vijay Joshi, Dombivli (IN); Manjunath Narayan Bhanu, Pune (IN)

Correspondence Address: Florek & Endres PLLC, 1156 Avenue of the Americas, Suite 600, New York, NY, 10036, US

(73) Assignee: WATSON PHARMA PRIVATE LIMITED, Andheri (East), Mumbai (IN)

(21) Appl. No.: 12/808,363

(22) PCT Filed: Dec. 18, 2008
(86) PCT No.: PCT/IN08/00845

§ 371 Date: Jun. 15, 2010

(30) Foreign Application Priority Data
Dec. 18, 2007 (IN) 2488/MUM/2007

(51) Int. Cl. C07B 57/00; C07C 227/42

(52) U.S. Cl. 562/401; 562/554

(57) ABSTRACT

The present invention provides a process for resolving racemic pregabalin to S-pregabalin by using a chiral acid to form a pregabalin salt, purifying the salt to obtain a purified S- pregabalin salt then neutralizing the salt to obtain S-pregabalin having good chiral purity.

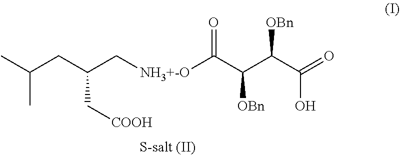

### FIELD OF THE INVENTION

The present invention relates to a process for preparation of S-Pregabalin, the optical resolution of 3-(aminomethyl)-5-methylhexanoic acid racemate [(±)-pregabalin], and the process for purification of salt of racemic pregabalin with a resolving agent such as Dibenzoyl-L-tartaric acid (II) and Di-p-toluoyl-D-tartaric acid.

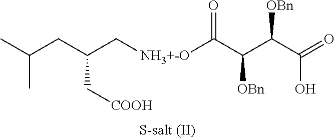

### BACKGROUND OF THE INVENTION

S-Pregabalin, S-(+)-3-(aminomethyl)-5-methylhexanoic acid (I) is an anticonvulsant and used in the treatment of epilepsy, pain, anxiety and social phobia. It may be represented by the chemical structure:

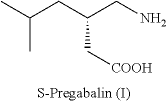

U.S. Pat. No. 5,637,767 describes method for manufacture of S-Pregabalin by combining racemic pregabalin with S-mandelic acid in water, an alcohol or a mixture of alcohol and water to precipitate a salt of S-pregabalin with S-mandelic acid. S-pregabalin is obtained by introducing the salt in polar aprotic solvent or a mixture of polar aprotic solvent and water. Alcohols used are methanol and isopropanol whereas polar aprotic solvents such as THF and DMSO are utilized.

U.S. Pat. No. 5,616,793 discloses method for preparation of S-pregabalin by optical resolution of (±)-3-(carbamoylmethyl)-5-methylhexanoic acid racemate (CMH-racemate). CMH-racemate is treated with R-phenethylamine in a mixture of solvent such as chloroform and ethanol to obtain R-CMH. R-CMH is then subjected to Hoffmann degradation with Br₂/NaOH to obtain S-pregabalin.

WO 2006/122259 A1 relates to the invention of optical resolution of CMH-racemate with an R-chiral amine such as ephedrine, ephedrine salt, norephedrine, and norephedrine salt. The R-CMH-ephedrine salt is then treated with HCl and worked up to obtain R-CMH.

US Patent Application Publication No. 2003/0212290 discloses asymmetric hydrogenation of a cyano-substituted olefin to obtain a cyano precursor of S-pregabalin. But the method describes use of CO₂ under pressure, which renders it difficult to be adaptable commercially.

US 2005283023A1 describes a method for preparing (S)-(+)-pregabalin and structurally-related compounds via enzymic kinetic resolution. Treatment of (R/S)-3-cyano-2-(ethoxycarbonyl)-5-methylhexanoic acid ethyl ester with lipase in buffered soln. of pH 8.0 afforded (S)-3-cyano-2-(ethoxycarbonyl)-5-methylhexanoic acid potassium salt and (R)-3-cyano-2-(ethoxycarbonyl)-5-methylhexanoic acid ethyl ester. Hydrogenation of the potassium salt over Raney Ni gave (S)-4-isobutyl-2-oxopyrrolidine-3-carboxylic acid, which was treated with aqueous HCl to afford pregabalin (80-85% yield, ee >99.5%).

WO 2005100580 A1 relates to regio-and stereoselective bioconversion of selected aliphatic dinitriles into corresponding cyanocarboxylic acids. Methods for the conversion of 2-isobutyl-succinonitrile into (S)-3-cyano-5-methylhexanoic acid, which is a useful intermediate in the synthesis of (S)-pregabalin is described.

Most of the above-mentioned patents are for the preparation of the resolved precursors which are eventually converted to S-pregabalin. Thus there has existed a need for a new resolution method that proceeds rapidly and efficiently, which can be industrially applicable and easy to scale up. The current invention satisfies this need by providing a method for the resolution of racemic pregabalin by converting into corresponding salt (S-salt) with a suitable chiral resolving agent followed by purification and liberation of pure S-pregabalin from the salt.

### OBJECT OF THE INVENTION

The object of present invention is to prepare S-pregabalin from racemic pregabalin.

Another object of the present invention is to prepare a salt of the racemic pregabalin with a suitable resolving agent. More particularly the object of the present invention is a process for the preparation of the pure S-pregabalin from S-salt of pregabalin.

### SUMMARY OF THE INVENTION

An embodiment of the invention provides a process for preparation of 5-pregabalin via optical resolution of racemic pregabalin with a suitable chiral acid resolving agent in a suitable solvent to obtain the corresponding S-salt followed by purification using a mixture of solvents to obtain S-salt of chiral purity>95%, preferably 97-99% and more preferably 98-99% as determined by HPLC.

In another embodiment, pure S-pregabalin is isolated from S-salt by further treating the S-salt of pregabalin with an acid and followed by an extraction step with a water immiscible solvent. The aqueous layer(s) is then treated with a base to obtain pure S-pregabalin.

### DETAILED DESCRIPTION OF THE INVENTION

The process for preparation of S-pregabalin from racemic pregabalin is schematically represented as follows:

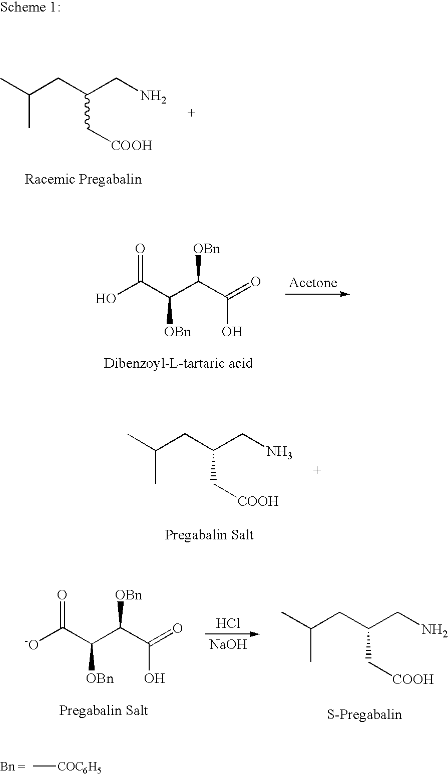

In an alternate embodiment of the present invention the dibenzoyl-L-tartaric acid in the above scheme may be replaced with di-p-toluoyl-D-tartaric acid or R-camphor sulfonic acid.

A further embodiment of the invention, the process for optical resolution of racemic pregabalin to S-pregabalin comprises:
i) treating racemic pregabalin with a suitable chiral resolving agent, such as a chiral acid resolving agent, in solvent such as a ketone, alcohol or mixtures thereof to form a mixture of S-salt and R-Salt;
ii) isolating the mixture of S-salt and R-Salt from the solvent;
iii) purifying said mixture of S-salt and R-Salt in solvent selected from a group consisting of ketones, alcohols, nitriles, ethers, water and mixtures thereof until S-pregabalin salt having a chiral purity of greater than 95%, S-pregabalin salt, preferably a chiral purity of greater than 97% and most preferably a chiral purity of greater than 98% is obtained.
v) neutralizing the purified S-pregabalin salt, and
vi) isolating the S-pregabalin

Preferred chiral resolving agents are Dibenzoyl-L-tartaric acid, Di-p-toluoyl-D-tartaric acid and R-Camphor Sulfonic acid. Most preferably, the chiral resolving agent is Dibenzoyl-L-tartaric acid or Di-p-toluoyl-D-tartaric acid. The resolution of racemic pregabalin is preferably performed in solvent comprising a C₂-C₆ ketone, most preferably in acetone. It can also be performed in solvent comprising C₁-C₅ alcohols, most preferably methanol.

Purification of S-pregabalin salt is preferably performed using a solvent selected from the group consisting of C₂-C₆ ketones, C₁-C₅ alcohols, nitriles, C₂-C₆ ethers, water and mixtures thereof. Most preferably the purification solvent comprises a mixture of C₂-C₆ ketones, C₁-C₅ alcohols and water such as an acetone-methanol mixture or an acetone-water mixture in the ratio's varying from 5-95%, most preferably 10-60%. One embodiment of the present invention requires suspending the crude S-pregabalin salt in a suitable solvent and refluxing the suspension. After refluxing, the reaction mass is cooled, filtered and washed. In a further embodiment of the present invention the purification step comprises two or more successive refluxing steps wherein each successive step employs a different purification solvent mixture of C₂-C₆ ketones, C₁-C₅ alcohols and water than the previous purification step.

Neutralizing of the S-pregabalin salt comprises treating the purified S-pregabalin salt with an acid, preferably an aqueous solution of a mineral acid. Examples of some of the mineral acids that may be used in the present invention include Hydrochloric acid, Sulfuric acid or Phosphoric acid, most preferably hydrochloric acid. Once a sufficient quantity of neutralizing acid has been reacted with the S-pregabalin salt, the liberated chiral resolving agent is removed from the neutralized material.

In one embodiment of the present invention, the liberated chiral resolving agent is removed by a solvent extraction process using an appropriate solvent system such as a solvent selected from C₃-C₆ esters, chlorinated solvents, aromatic hydrocarbons, ethers and mixtures thereof, most preferably using ethyl acetate. Isolation of S-pregabalin from the resulting aqueous solution may be performed by adjusting the pH of the neutralized S-pregabalin to a pH of about 3 to about 6, preferably about a pH of about 4 to about 5 and most preferably a pH of about 4.6 to about 4.7. In an alternate embodiment, the pH of the neutralized S-pregabalin solution is adjusted until the S-pregabalin precipitates. In one embodiment of the present invention, the pH of the neutralized S-pregabalin solution is adjusted using a base such as alkali or alkaline earth metal hydroxides, alkali or alkaline earth metal bicarbonates, alkali or alkaline earth metal carbonates or organic amines, most preferably sodium hydroxide or potassium hydroxide.

The S-pregabalin prepared in accordance with the present invention may be mixed with at least one additional conventional pharmaceutical excipient to prepare a pharmaceutical dosage form such as a tablets, capsule or solution. The pharmaceutical dosage form may be administered to patients to treat epilepsy, pain, anxiety and social phobia.

The following examples illustrate various aspects of the present invention:

### Example 1

Resolution of Racemic Pregabalin with Dibenzoyl-L-tartaric Acid:

Racemic pregabalin, 200 gm was suspended in 1000 ml of acetone. To the slurry was added Dibenzoyl-L-tartaric acid (472 gm) dissolved in 1000 ml of acetone at 25-30° C. The reaction mass was stirred at 25-30° C. for 1 h. The reaction mass was then filtered and the solid obtained was washed twice with 200 ml acetone. The product was dried in an oven at 70° C. Dry wt. of S-salt=291 gin with chiral purity of 97.15%.

### Example 2

### Purification of S-pregabalin Salt

The crude S-pregabalin salt of Example 1, 290 gm was suspended in 2000 ml of an acetone-methanol mixture (9:1). The reaction mass was heated to reflux and maintained for 30 min. The reaction mass was then cooled to 25-30° C. and maintained for 45-60 min. The reaction mass was then filtered and the solid obtained was washed twice with 200 ml acetone-methanol mixture (9:1). The isolated product was dried in an oven at 70° C. Dry wt. of the S-pregabalin salt=270 gm with chiral purity of 98.2%.

### Example 3

### Purification of S-pregabalin Salt

The S-pregabalin salt of Example 2, 10 gm was suspended in 70 ml of an acetone-methanol mixture (4:1). The reaction mass was then heated to reflux and maintained for 30 min. The reaction mass was then cooled to 25-30° C. and maintained for 45-60 min. The reaction mass was then filtered and the solid obtained was washed twice with 10 ml of an acetone-methanol mixture (4:1). The isolated product was dried in an oven at 70° C. Dry wt. of the S-pregabalin salt=9.5 gm with chiral purity of 97.08%.

### Example 4

### Purification of S-pregabalin Salt

The S-pregabalin salt of Example 2, 10 gm was suspended in 70 ml of acetone-methanol mixture (7:3). The reaction mass was then heated to reflux and maintained for 30 min. The reaction mass was then cooled to 25-30° C. and maintained for 45-60 min. The reaction mass was then filtered and the solid obtained was washed twice with 10 ml of an acetone-methanol mixture (7:3). The isolated product was dried in an oven at 70° C. Dry wt. of the S-pregabalin salt=8.9 gm with chiral purity of 97.78%.

### Example 5

### Purification of S-pregabalin Salt

The S-pregabalin salt of Example 2, 10 gm was suspended in 70 ml of an acetone-methanol mixture (3:2). The reaction mass was then heated to reflux and maintained for 30 min. The reaction mass was then cooled to 25-30° C. and maintained for 45-60 min. The reaction mass was then filtered and the solid obtained was washed twice with 10 ml of an acetone-methanol mixture (3:2). The isolated product was dried in an oven at 70° C. Dry wt. of the S-pregabalin salt=8.2 gm with chiral purity of 97.89%.

### Example 6

Resolution of Racemic Pregabalin with Di-p-toluoyl-D-tartaric Acid:

Racemic pregabalin, 5.0 gm was suspended in 25 ml methanol. To the slurry was added Di-p-toluoyl-D-tartaric acid (12.1 gm) dissolved in 25 ml of acetone at 25-30° C. The reaction mass was stirred at 25-30° C. for 1 h. The reaction mass was then filtered and the solid obtained was washed twice with 5 ml of a methanol-acetone mixture (1:1). The isolated product was dried in an oven at 70° C. Dry wt. of the S-pregabalin salt=7.2 gm with chiral purity of 84.60%.

### Examples 7

### Purification of S-pregabalin Salt

The crude S-pregabalin salt of Example 6, 5.0 gm was suspended in 50 ml of a methanol-acetone mixture (7:3). The reaction mass was then heated to reflux and maintained for 30 min. The reaction mass was then cooled to 25-30° C. and maintained for 1 h. The reaction mass was then filtered and the solid obtained was washed twice with 5 ml of a methanol-acetone mixture (7:3). The isolated product was dried in an oven at 70° C. Dry wt. of the S-pregabalin salt=2.6 gm with chiral purity of 95.35%.

### Example 8

### Purification of S-pregabalin Salt

The crude S-pregabalin salt of Example 6, 5.0 gm was suspended in 50 ml an acetone-water mixture (19:1). The reaction mass was then heated to reflux and maintained for 30 min. The reaction mass was then cooled to 25-30° C. & maintained for 1 h. The reaction mass was then filtered and the solid obtained was washed twice with 5 ml of an acetone-water mixture (19:1). The isolated product was dried in an oven at 70° C. Dry wt. of the S-pregabalin salt=4.5 gm with chiral purity of 98.64%.

### Example 9

### Purification of S-pregabalin Salt

The crude S-pregabalin salt of Example 6, 5.0 gm was suspended in 50 ml of an acetone-water mixture (9:1). The reaction mass was then heated to reflux and maintained for 30 min. The reaction mass was then cooled to 25-30° C. and maintained for 1 h. The reaction mass was then filtered and the solid obtained was washed twice with 5 ml of an acetone-water mixture (9:1). The isolated product was dried in an oven at 70° C. Dry wt. of the S-pregabalin salt=4.3 gm with chiral purity of 98.93%.

### Example 10

### Preparation of S-pregabalin

The S-pregabalin salt of Example 2, 10gm was added to 10 ml of 10% HCl solution. The reaction mass was heated to 50-55° C. and maintained for 30 min. The reaction mass was then cooled to 25-30° C. and 20 ml of ethyl acetate was added. The reaction mixture was stirred for 15 min. and was then subjected to a layer separation. The aqueous layer was further extracted with 10 ml of ethyl acetate. The aqueous layer thus obtained was then cooled to 5-10° C. The pH of the aqueous solution was adjusted to 4.6-4.7 with a 20% NaOH solution and maintained at 5-10° C. for 45-60 min. The reaction mass was then filtered and the solid obtained was washed twice with 5 ml of chilled water. The product was dried in an oven at 70° C. Dry wt. of the S-Pregabalin=1.8 gm (SOR=10.835, Chiral purity—98.86% S).

Present invention has been particularly described, in conjunction with a specific preferred embodiment and it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

1. A process for optical resolution of racemic pregabalin comprising:
a) treating racemic pregabalin with a chiral acid resolving agent in a first solvent to obtain a corresponding salt,
b) isolating the salt and
c) purifying the salt in a second solvent selected from the group consisting of ketones, alcohols, nitriles, ethers, water and mixtures thereof

2. The process of claim 1, wherein the salt has a chiral purity of greater than about 95 percent by HPLC.

3. The process of claim 1, wherein the salt has a chiral purity of about 98%-99% area by HPLC.

4. The process of claim 1, wherein the resolving agent is dibenzoyl-L-tartaric acid or di-p-toluoyl-D-tartaric acid.

5. The process of claim 1, wherein the first solvent is acetone or methanol.

6. The process of claim 1, wherein the second solvent is selected from the group consisting of acetone-methanol, acetone-water and mixtures thereof.

7. A process for preparation of S-pregabalin comprising:
a) combining a purified S-pregabalin salt with a mineral acid to form a solution and extracting the liberated chiral resolving agent in solvents selected from the group consisting of esters, chlorinated solvents, aromatic hydrocarbons, ethers and mixtures thereof,
b) separating an aqueous layer from the solution,
c) adjusting the pH of the separated aqueous layer to about 4-5 with a base to obtain a precipitate and
d) isolating the precipitate.

8. The process of claim 7, wherein the solvent for extraction is ethyl acetate.

9. The process of claim 7, wherein the mineral acid is aqueous hydrochloric acid.

10. The process of claim 7, wherein 10% aqueous hydrochloric acid is used.

11. The process of claim 7, wherein the base is aqueous sodium hydroxide.

12. The process of claim 7, wherein the pH is adjusted to about 4.6-4.7.
